(19) United States
(12) Patent Application Publication
(10) Pub. No.: US 2024/0052292 A1
(43) Pub. Date: Feb. 15, 2024

(54) CELL MANAGEMENT SYSTEM AND CELL MANAGEMENT METHOD

(71) Applicants: CANON MEDICAL SYSTEMS CORPORATION, Tochigi (JP); Canon Kabushiki Kaisha, Tokyo (JP)

(72) Inventors: Mayumi SAKAGAMI, Ibaraki (JP); Koji HIRATA, Kasukabe (JP)

(73) Assignees: CANON MEDICAL SYSTEMS CORPORATION, Tochigi (JP); Canon Kabushiki Kaisha, Tokyo (JP)

(21) Appl. No.: 18/447,437

(22) Filed: Aug. 10, 2023

(30) Foreign Application Priority Data
Aug. 12, 2022 (JP) 2022-128677

(51) Int. Cl. C12M 1/00; C12M 1/36

(52) U.S. Cl. CPC C12M 47/04; C12M 41/48

(57) ABSTRACT

A cell management system according to an embodiment includes an analysis apparatus and a determination apparatus. The analysis apparatus acquires a first identification code from a processing sample from which a cell used for treatment is produced, or from a processed product based on the processing sample, and acquires a second identification code from a reference sample. Based on the first identification code and the second identification code, the determination apparatus determines the consistency of a subject from whom the processing sample is obtained and a subject from whom the reference sample is obtained.

### CROSS-REFERENCE TO RELATED APPLICATIONS

This application is based upon and claims the benefit of priority from Japanese Patent Application No. 2022-128677, filed on Aug. 12, 2022; the entire contents of which are incorporated herein by reference.

### FIELD

Embodiments described herein relate generally to a cell management system and a cell management method.

### BACKGROUND

In recent years, personalized medicine using cells has become increasingly popular. As a specific personalized medicine, research has been developed on an autologous cell therapy, regenerative medicine, prior drug response evaluation, and disease response evaluation. In the personalized medicine, cell management is important because many kinds and small quantity of cells may be produced during cell processing.

### BRIEF DESCRIPTION OF THE DRAWINGS

FIG. 1 is a block diagram illustrating an example of a configuration of a cell management system according to a first embodiment;

FIG. 2 is a flowchart illustrating an outline of a processing procedure by a cell management system according to the first embodiment;

FIG. 3 is a diagram illustrating an example of cell management according to the first embodiment;

FIG. 4 is a diagram illustrating an example of cell management according to the first embodiment;

FIG. 5A is a diagram illustrating a workflow according to the first embodiment;

FIG. 5B is a diagram illustrating a workflow according to the first embodiment;

FIG. 6 is a diagram illustrating a workflow related to the acquisition of an identification code according to the first embodiment;

FIG. 7 is a diagram illustrating an example of a process performed by the cell management system according to the first embodiment;

FIG. 8 is a diagram illustrating an example of a process performed by the cell management system according to the first embodiment; and

FIG. 9 is a display example according to the first embodiment.

### DETAILED DESCRIPTION

A cell management system according to embodiments includes an analysis apparatus configured to acquire a first identification code from a processing sample from which a cell used for treatment is produced, or from a processed product based on the processing sample, and to acquire a second identification code from a reference sample; and a determination apparatus, based on the first identification code and the second identification code, configured to determine consistency of a subject from whom the processing sample is obtained and a subject from whom the reference sample is obtained.

Hereinafter, embodiments of a cell management system and a cell management method will be described in detail with reference to the accompanying drawings.

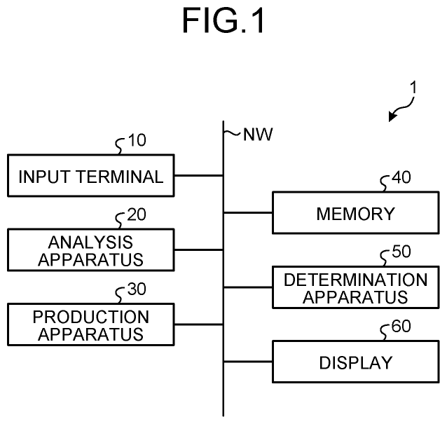

FIG. 1 is a configuration example of a cell management system 1. The cell management system 1 in FIG. 1 includes an input terminal 10, an analysis apparatus 20, a production apparatus 30, a memory 40, a determination apparatus 50, and a display 60.

The input terminal 10 is an interface that receives input of various types of information from a user of the cell management system 1. For example, the input terminal 10 receives an input of information such as subject information, sample information, and processed product information, which will be described below.

Specifically, the input terminal 10 converts an input operation performed by the user into an electrical signal, and transmits the converted electrical signal to the memory 40 to store. For example, the input terminal 10 is implemented by a trackball, a switch button, a mouse, a keyboard, a touch pad with which input operations are performed by touching an operation surface, a touch screen in which a display screen and a touch pad are integrated, a non-contact input interface using an optical sensor, a voice input interface, and the like. The input terminal 10 is not limited to the one provided with physical operation parts such as a mouse. For example, an example of the input terminal 10 also includes a processing circuit that receives an electrical signal corresponding to an input operation from an external input device, which is provided separately from the device, and that transmits the electrical signal to the memory 40.

The analysis apparatus 20 is an apparatus that acquires an identification code, by analyzing a processing sample X1, a reference sample Y1, or a processed product based on the processing sample X1. By comparing the identification codes, it is possible to determine whether the subjects from whom the identification codes are obtained are the same. The analysis apparatus 20 is an example of an acquisition unit. The analysis apparatus 20 includes a processing circuit, and implements a function corresponding to a computer program, by reading and executing the computer program as appropriate. Details of the processing performed by the analysis apparatus 20 will be described below.

The production apparatus 30 is an apparatus that produces various processed products from the processing sample X1. For example, blood of the subject is collected as the processing sample X1. In this example, the production apparatus 30 can produce blood-derived CD34-positive cells, blood-derived iPS cells, and iPS cell-derived cardiomyocytes.

The memory 40 stores various types of information. For example, the memory 40 stores the subject information, sample information, and processed product information, input via the input terminal 10. Moreover, the memory 40 stores an identification code obtained by the analysis apparatus 20. For example, the memory 40 is implemented by a semiconductor memory element such as a Read Only Memory (ROM), a Random Access Memory (RAM), and a flash memory, a hard disk, an optical disc, and the like. The memory 40 may also be implemented by a cloud computer connected to the cell management system 1 via a network NW.

For example, the memory 40 has database 41 and database 42. Data on the processing sample X1 will be registered in the database 41. For example, an identification code CX1 obtained from the processing sample X1, and an identification code obtained from the processed product based on the processing sample X1 will be registered in the database 41. Data on the reference sample Y1 will be registered in the database 42. For example, an identification code CY1 obtained from the reference sample Y1 will be registered in the database 42.

The determination apparatus 50 determines the consistency of the subject from whom the processing sample X1 is obtained and the subject from whom the reference sample Y1 is obtained, by comparing the identification code registered in the database 41 and the identification code registered in the database 42. The determination apparatus 50 is an example of a determination unit. The determination apparatus 50 includes a processing circuit, and implements the function corresponding to a computer program, by reading and executing the computer program as appropriate. Details of the processing performed by the determination apparatus 50 will be described below.

The display 60 displays the determination results of the determination apparatus 50. That is, the display 60 displays whether the subject from whom the processing sample X1 is obtained and the subject from whom the reference sample Y1 is obtained are the same. The display 60 is implemented by a liquid crystal display, a Cathode Ray Tube (CRT) display, or the like.

For example, the processing circuit described above is implemented by a processor. The processing circuit can also be configured by combining a plurality of independent processors, and each processor can execute a computer program to implement each processing function.

A configuration example in FIG. 1 is merely an example, and the configuration of the cell management system 1 may be changed as appropriate. For example, the analysis apparatus 20 and the determination apparatus 50 may be implemented by one processing circuit executing various types of computer programs. Moreover, for example, the input terminal 10 and the display 60 can be integrated into a tablet terminal, a smartphone terminal, and the like provided with a touch panel.

Furthermore, FIG. 1 illustrates an example in which the various components included in the cell management system 1 are connected to each other via a network NW. However, not all components need to be connected to each other. For example, the input terminal 10 need not be directly connected to the memory 40. If the input terminal 10 is connected to one of the components such as the analysis apparatus 20, the information input from the input terminal 10 can be stored in the memory 40.

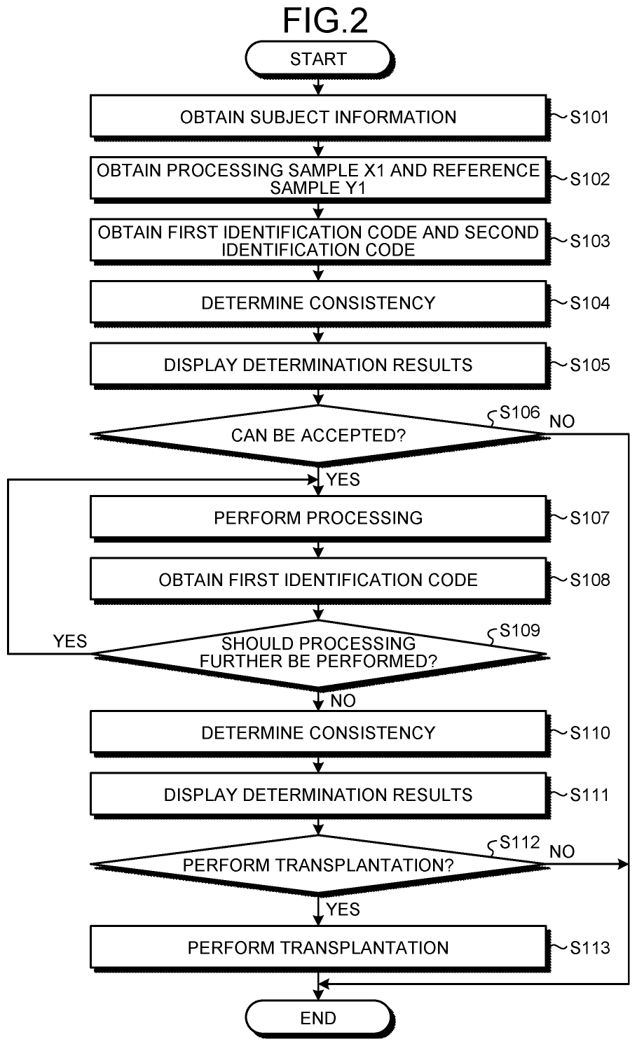

Next, the flow of processing performed by the cell management system 1 will be described in detail, with reference to the flowchart in FIG. 2. FIG. 2 is a flowchart illustrating an outline of a processing procedure by the cell management system 1 according to the first embodiment.

First, the cell management system 1 obtains subject information (step S101). In this example, the subject information is information on the subject himself/herself. The specific examples of the subject information include a subject's ID, disease name, past medical history, family history, and the like. For example, the cell management system 1 receives an input operation of the subject information from a user such as a doctor via the input terminal 10, and causes the memory 40 to store the received subject information. Alternatively, the cell management system 1 may obtain the subject information registered in advance via the network NW, and cause the memory 40 to store the subject information. For example, if subject information is already registered in the Hospital Information System (HIS), the cell management system 1 can obtain the subject information by accessing the HIS.

Next, a user such as a doctor obtains the processing sample X1 and the reference sample Y1 from the subject (step S102). The user then enters sample information. In this example, the processing sample X1 and the reference sample Y1 are different types of samples obtained from the same subject. For example, if the processing sample X1 is the blood of a subject, the reference sample Y1 is the oral cells obtained from the same subject.

Alternatively, the processing sample X1 and the reference sample Y1 may be the same type of samples obtained from the same subject in different tests. For example, if the processing sample X1 is the blood of the subject, the reference sample Y1 is the blood obtained from the same subject on a different day. For example, the processing sample X1 and the reference sample Y1 are not limited to blood and oral cells, but may be changed to skin, hair, or the like as appropriate.

Next, the cell management system 1 acquires a first identification code from the processing sample X1, and acquires a second identification code from the reference sample Y1 (step S103). For example, as the first identification code, the analysis apparatus 20 obtains an identification code CX1 from the processing sample X1, and causes the memory 40 to store the identification code CX1. Moreover, as the second identification code, the analysis apparatus 20 obtains an identification code CY1 from the reference sample Y1, and causes the memory 40 to store the identification code CY1.

Next, the cell management system 1 determines the consistency of the subject from whom the processing sample X1 is obtained and the subject from whom the reference sample Y1 is obtained (step S104). Specifically, the determination apparatus 50 determines the consistency of the subjects, by comparing the identification code CX1 and the identification code CY1 obtained at step S103. Moreover, the display 60 displays the determination results at step S104 (step S105).

In this example, the cell management system 1 determines whether the processing sample X1 can be accepted (step S106). Specifically, if it is displayed that the subject from whom the processing sample X1 is obtained and the subject from whom the reference sample Y1 is obtained are not the same at step S105, the processing is terminated without accepting the processing sample X1 (No at step S106).

On the other hand, if it is displayed that the subject from whom the processing sample X1 is obtained and the subject from whom the reference sample Y1 is obtained are the same at step S105, the production apparatus 30 performs processing on the processing sample X1 to produce a processed product (step S107). In this example, the user enters processed product information. Moreover, the analysis apparatus 20 obtains the identification code based on the processed product produced at step S107 as the first identification code (step S108).

In this example, the cell management system 1 determines whether the processing at step S107 should further be performed (step S109). For example, if iPS cells or cardiomyocytes are to be produced after the production of CD34-positive cells, it is determined that the processing will further be performed (Yes at step S109), and the processes at step S107 and step S108 will be repeated. On the other hand, if the processing will not be performed (No at step S109), the consistency is determined at step S110.

In this process, if the processes at step S107 and step S108 are repeated, multiple first identification codes based on the processed product will be obtained. For example, if the CD34-positive cells, iPS cells, and cardiomyocytes are produced, three first identification codes are obtained. At step S110, the determination apparatus 50 determines the consistency of the subjects, based on the multiple first identification codes and the second identification code obtained at step S103. It is possible to determine the consistency of the subjects by only acquiring one first identification code. However, it is possible to improve the determination accuracy by acquiring multiple first identification codes.

Next, the display 60 displays the determination results at step S110 (step S111). In this example, the cell management system 1 determines whether to perform transplantation (step S112). Specifically, if it is displayed that the subject from whom the processing sample X1 is obtained and the subject from whom the reference sample Y1 is obtained are not the same at step S111, the process is terminated without performing transplantation (No at step S112).

On the other hand, if it is displayed that the subject from whom the processing sample X1 is obtained and the subject from whom the reference sample Y1 is obtained are the same at step S111, it is determined that transplantation will be performed (Yes at step S112), and part or all of the processed products produced at step S107 will be transplanted into the subject (step S113). For example, if the CD34-positive cells, iPS cells, and cardiomyocytes are produced at step S107, the transplantation of CD34-positive cells and cardiomyocytes will be carried out.

Because the processing sample X1 and the reference sample Y1 are acquired from the same subject, the determination made at step S106 and step S112 should be basically the “same”. However, in clinical settings, due to various factors, the determination may be made as “not the same”.

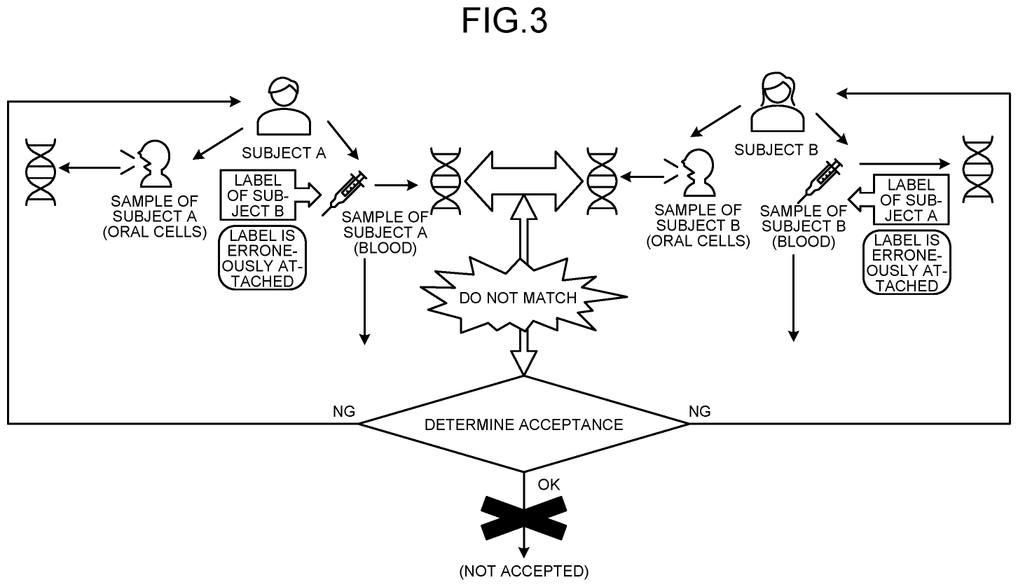

FIG. 3 illustrates an example in which the determination is made as “not the same”. In FIG. 3, blood and oral cells are collected as samples, from the subject A and the subject B. Moreover, the collected samples are labeled for management. In this example, labeling is basically performed manually. Hence, for example, there may be a case when a label indicating the subject B may be erroneously attached to the sample (blood) of the subject A.

However, in the cell management system 1 described above, it is possible to detect that a label is erroneously attached. That is, if the blood of the subject A is the processing sample X1, the flow in FIG. 2 proceeds while using the oral cells of the subject B as the reference sample Y1. Hence, at step S105, it is displayed that the subject from whom the processing sample X1 is obtained and the subject from whom the reference sample Y1 is obtained are not the same. Then, in the determination at step S106, for example, it is possible to obtain the sample again, without performing the steps subsequent to step S107.

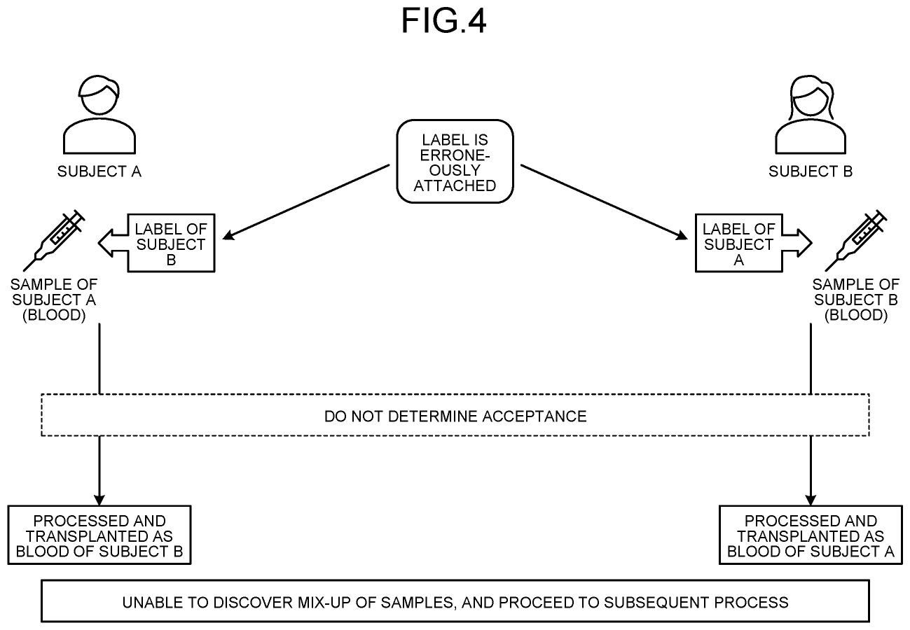

On the other hand, if the consistency of the subjects is not determined as described above, and if the acceptance of the sample will not be determined, as illustrated in FIG. 4, the process will proceed to the subsequent step without discovering the mix-up of the samples. This could result in transplanting the processed product based on the cells of a different subject, or performing unnecessary treatments. In contrast, according to the cell management system 1, it is possible to detect the mix-up of samples, and implement reliable cell management. That is, with the cell management system 1, it is possible to implement reliable cell management, by directly managing the collected samples and the cells of the processed products themselves, instead of indirect management methods using a means for managing the attachment of a label such as a barcode onto a container, a means for managing by human leukocyte antigen, and a means for managing culture history of a cultivator.

In addition to the mix-up of samples at the time of sample collection illustrated in FIG. 3 and FIG. 4, for example, various other failures may also occur during the cell processing process. For example, other failures may include situations such as a mix-up of samples during the manual-handling tasks, a mix-up due to transferring wrong information onto a plate, a mix-up due to transferring wrong information when cryovial is stored, information loss due to a failure in the automation system, and the like. Even in these cases, the cell management system 1 can detect the mix-up of samples, and implement reliable cell management.

Another method that directly manages the samples and the cells of the processed products themselves includes a method in which a DNA sequence for identification is incorporated into the genomic DNA of the cells, and use the obtained result as an identification code. Specifically, barcode sequence addition to a fragment, an amplified DNA sequence, and the like may be used for the cell management as an identification code. However, because the method involves genetic modification, measures must be taken from the viewpoint of safety, when the cells are used for transplantation. In contrast, in the cell management system 1, the DNA sequence existing in a cell is used as the identification code. Hence, it is possible to implement reliable cell management without concerning about the safety.

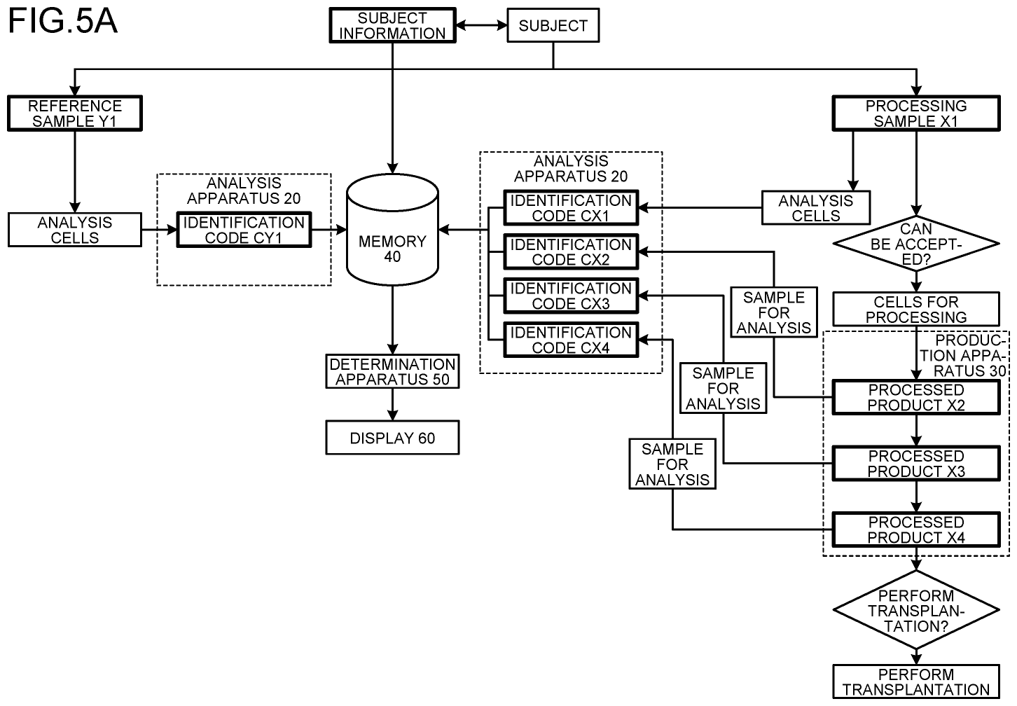

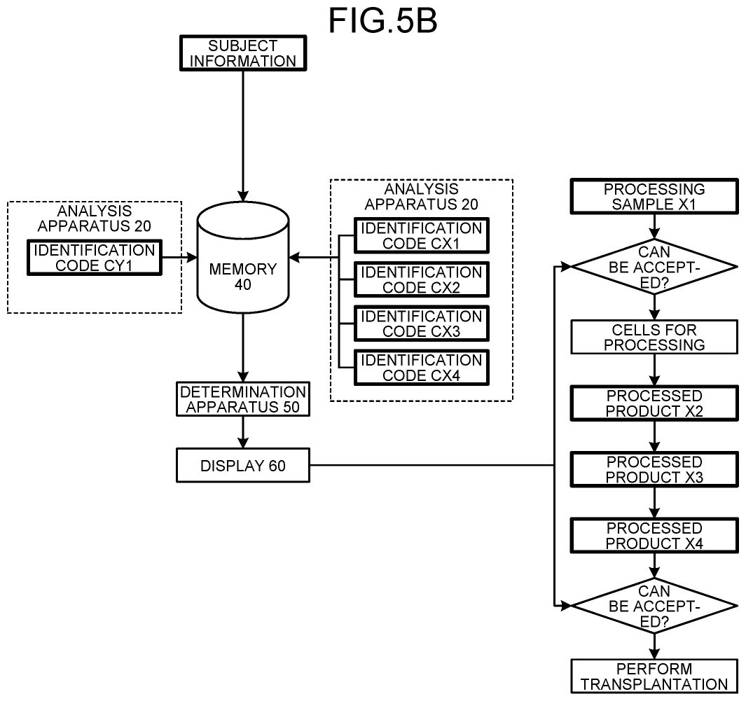

Next, with reference to FIG. 5A and FIG. 5B, a series of workflows until the processed cells are transplanted will be described.

As described above, the subject information is obtained, and stored in the memory 40. For example, the user operates the input terminal 10 to enter the subject information such as the subject's ID, disease name, the past medical history, family history, and the like. The input data are stored in the memory 40.

Moreover, as illustrated in FIG. 5A, the processing sample X1 and the reference sample Y1 are obtained from the subject. For example, blood is collected as the processing sample X1, and oral cells are collected as the reference sample Y1, from the same subject.

The analysis apparatus 20 analyzes a part of the processing sample X1 as analysis cells, and obtains the identification code CX1. The identification code CX1 is an example of the first identification code. Moreover, the analysis apparatus 20 analyzes part or all of the reference sample Y1 as analysis cells, and obtains the identification code CY1. The identification code CY1 is an example of the second identification code. The identification code CX1 and the identification code CY1 are stored in the memory 40.

The determination apparatus 50 reads the identification code CX1 and the identification code CY1 from the memory 40 to compare, and determines the consistency of the subject from whom the processing sample X1 is obtained and the subject from whom the reference sample Y1 is obtained. The display 60 displays the determination results of the determination apparatus 50. As illustrated in FIG. 5B, according to the display of the determination results, whether the processing sample X1 is acceptable will be determined. For example, if the displayed determination results indicate that the subjects are not the same, the processing sample X1 and the reference sample Y1 are obtained again.

On the other hand, if the displayed determination results indicate that the subjects are the same, the processing sample X1 is accepted, and the production apparatus 30 starts processing the processing sample X1. Specifically, the production apparatus 30 produces a processed product using a part of the processing sample X1 that was not used for analysis by the analysis apparatus 20 (cells for processing).

In this example, a plurality of processed products based on the processing sample X1 may be produced. For example, CD34-positive cells, iPS cells, and cardiomyocytes may be each produced, based on the blood collected as the processing sample X1. As illustrated in FIG. 5A, the following describes the case when a processed product X2, a processed product X3, and a processed product X4 will be produced, based on the processing sample X1.

When the processed product X2 is produced, the analysis apparatus 20 analyzes a part of the processed product X2 as a sample for analysis, and obtains an identification code CX2. Similarly, when the processed product X3 is produced, the analysis apparatus 20 analyzes a part of the processed product X3 as a sample for analysis, and obtains an identification code CX3. Similarly, when the processed product X4 is produced, the analysis apparatus 20 analyzes a part of the processed product X4 as a sample for analysis, and obtains an identification code CX4. The memory 40 stores the identification code CX2, the identification code CX3, and the identification code CX4. The identification code CX2, the identification code CX3, and the identification code CX4 are examples of the first identification code.

Moreover, the determination apparatus 50 reads the identification code CX2, the identification code CX3, the identification code CX4, and the identification code CY1 from the memory 40 to compare, and determines the consistency of the subject from whom the processed product X2 is obtained and the subject from whom the reference sample Y1 is obtained. Furthermore, the display 60 displays the determination results of the determination apparatus 50. Then, if the displayed determination results indicate that the subjects are the same, transplantation will be carried out on the subject.

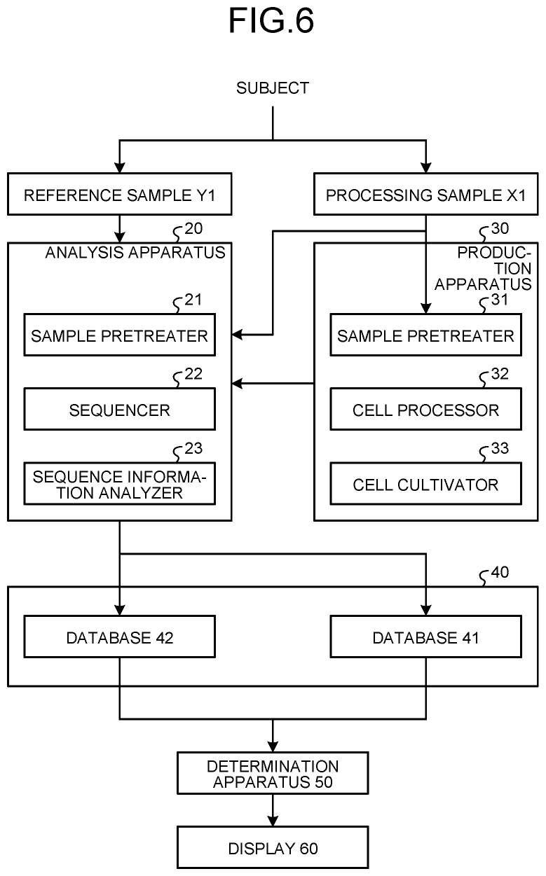

Next, the acquisition of the identification code will be described with reference to FIG. 6. FIG. 6 is a diagram illustrating a workflow related to the acquisition of an identification code.

As illustrated in FIG. 6, the analysis apparatus 20 performs analysis using a part of the processing sample X1 obtained from the subject. Moreover, the analysis apparatus 20 performs analysis using a part of the processed product based on the processing sample X1. Furthermore, the analysis apparatus 20 performs analysis using a part or all of the reference sample Y1 obtained from the subject. For example, as illustrated in FIG. 6, the analysis apparatus 20 includes a sample pretreater 21, a sequencer 22, and a sequence information analyzer 23.

For example, the sample pretreater 21 performs sample pretreatment on the processing sample X1. In this example, the sample to be pretreated is a part of the processing sample X1. The sequencer 22 analyzes the processing sample X1 after the sample pretreatment, obtains the gene sequence, and obtains cell identification base information BX1 from the obtained gene sequence. The sequence information analyzer 23 obtains the identification code CX1 based on the processing sample X1. In this example, the sequence information analyzer 23 can obtain the identification code CX1 by processing the cell identification base information BX1, or can obtain the identification code CX1 by analyzing the processing sample X1 after the sample pretreatment. The processing results of the analysis apparatus 20 such as the cell identification base information BX1 and the identification code CX1, are stored in the database 41 of the memory 40.

Similarly, the sample pretreater 21 performs sample pretreatment on the reference sample Y1. In this example, the sample to be pretreated is a part or all of the reference sample Y1. The sequencer 22 obtains the cell identification base information BY1, by analyzing the reference sample Y1 after the sample pretreatment. The sequence information analyzer 23 obtains the identification code CY1 based on the reference sample Y1. In this example, the sequence information analyzer 23 can obtain the identification code CY1 by processing the cell identification base information BY1, or can obtain the identification code CY1 by analyzing the reference sample Y1 after the sample pretreatment. The processing results of the analysis apparatus 20 such as the cell identification base information BY1 and the identification code CY1, are stored in the database 42 of the memory 40.

The determination apparatus 50 obtains the identification code CX1 from the database 41, and obtains the identification code CY1 from the database 42. For example, the determination apparatus 50 obtains various identification codes, triggered by a request from the user. Moreover, on the basis of the obtained identification codes, the determination apparatus 50 determines the consistency of the subject from whom the processing sample X1 is obtained and the subject from whom the reference sample Y1 is obtained. Furthermore, the display 60 displays the determination results of the determination apparatus 50 to determine whether the processing sample X1 is acceptable.

The production apparatus 30 produces a processed product using a part of the processing sample X1 that was not used for analysis by the analysis apparatus 20 (cells for processing). For example, the production apparatus 30 produces the processed product X2, the processed product X3, and the processed product X4 described above. For example, the production apparatus 30 includes a sample pretreater 31, a cell processor 32 and a cell cultivator 33 as illustrated in FIG. 6, and performs processes such as sample pretreatment, process and culture for cells for processing

When the processed product X2 is produced, the sample pretreater 21 performs sample pretreatment using a part of the processed product X2 as a sample for analysis. The sequence information analyzer 23 obtains the identification code CX2, by analyzing the processed product X2 after the sample pretreatment. In this example, the sequencer 22 may obtain the cell identification base information BX2 by analyzing the reference sample X2 after the sample pretreatment. Alternatively, the acquisition of the cell identification base information BX2 may be omitted. Similarly, the sequence information analyzer 23 obtains the identification code CX3, by analyzing the processed product X3 after the sample pretreatment. Similarly, the sequence information analyzer 23 obtains the identification code CX4, by analyzing the processed product X4 after the sample pretreatment. The processing results by the analysis apparatus 20 such as the identification code CX2, the identification code CX3, and the identification code CX4, are stored in the database 41 of the memory 40.

The determination apparatus 50 obtains the identification code CX2, the identification code CX3, and the identification code CX4 from the database 41, and obtains the identification code CY1 from the database 42. On the basis of the obtained identification codes, the determination apparatus 50 determines the consistency of the subject from whom the processed product X2 is obtained and the subject from whom the reference sample Y1 is obtained. Moreover, the display 60 displays the determination results of the determination apparatus 50 to determine whether transplantation is possible.

In this example, the cell identification base information BX1 and the cell identification base information BY1 are sequence information such as genomic DNA sequence. The cell identification base information BX1 and the cell identification base information BY1 are large-sized data obtained by detailed analysis.

On the other hand, for example, the identification code CX1, the identification code CX2, the identification code CX3, the identification code CX4, and the identification code CY1 are short tandem repeat (STR) sequences, multi-locus minisatellite (MLmS) sequences, single-locus minisatellite (SLmS) sequences, mitochondrial DNA sequences, and the like. The identification code such as the identification code CX1 is small-sized data that can be easily obtained.

The analysis apparatus 20 may obtain only one type of the identification code such as the identification code CX1 or multiple types thereof. For example, as the identification code such as the identification code CX1, the analysis apparatus 20 may only obtain the STR sequence or may obtain the STR sequence and the MLmS sequence. The type of the identification code to be obtained may be set in advance, or may be set by the user as appropriate.

In the determination apparatus 50, for example, it is also possible to determine the consistency of the subject from whom the processing sample X1 is obtained and the subject from whom the reference sample Y1 is obtained, by comparing between the cell identification base information BX1 and the cell identification base information BY1. However, it takes time to obtain the genomic DNA sequence and the like, and also, because the data size is large, the processing load will increase. In particular, in a case where a plurality of processed products are generated, if the genomic DNA sequence or the like is obtained from each of the processed products, convenience (efficiency) is sacrificed.

In contrast, the identification code such as the STR sequence can be obtained in a short time, and can be processed with a small load. Moreover, as described above, the cell management system 1 implements reliable cell management, by directly managing the collected samples and the cells of the processed products themselves. In other words, the cell management system 1 can implement simple and reliable cell management, by performing a process limited to a part that can specify the individual in the entire DNA sequence. In particular, personalized medicine that has become increasing popular in recent years has the characteristics of many kinds and small quantity. In the personalized medicine, it is important to prevent a mix-up without fail, and implement the prevention process in a simple manner.

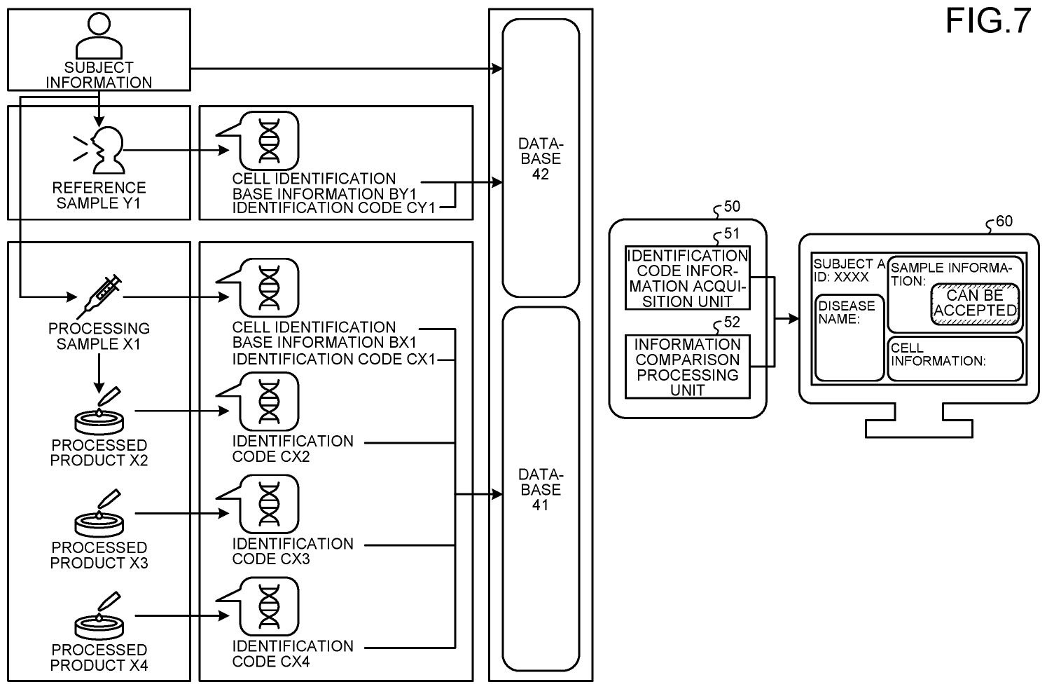

Next, the processing of the cell management system 1 will be described with reference to FIG. 7. First, the subject information is entered via the input terminal 10 or the like, and the input subject information is registered in the database 41 and the database 42.

Moreover, the processing sample X1 is collected from the subject, and the cell identification base information BX1 and the identification code CX1 are obtained from the processing sample X1. The cell identification base information BX1 and the identification code CX1 are registered in the database 41, after being associated with the subject information and the sample information. Moreover, the reference sample Y1 is collected from the subject, and the cell identification base information BY1 and the identification code CY1 are obtained from the reference sample Y1. The cell identification base information BY1 and the identification code CY1 are registered in the database 42, after being associated with the subject information and the sample information. The acquisition of the cell identification base information BX1 and the cell identification base information BY1 may be omitted as appropriate.

Moreover, based on the processing sample X1, the processed product X2, the processed product X3, and the processed product X4 will be produced. Furthermore, the identification code CX2, the identification code CX3, and the identification code CX4 are each obtained, based on the processed product X2, the processed product X3, and the processed product X4, and are registered in the database 41 after being associated with the subject information and the processed product information.

The determination apparatus 50 determines the consistency of the subject from whom the processing sample X1 is obtained and the subject from whom the reference sample Y1 is obtained, based on the first identification code and the second identification code. For example, as illustrated in FIG. 7, the determination apparatus 50 includes an identification code information acquisition unit 51 and an information comparison processing unit 52.

The identification code information acquisition unit 51 acquires the identification codes from the database 41 and the database 42 as appropriate. For example, to perform the determination at step S104 in FIG. 2, the identification code information acquisition unit 51 acquires the identification code CX1 from the database 41, and acquires the identification code CY1 from the database 42. Moreover, to perform the determination at step S110 in FIG. 2, the identification code information acquisition unit 51 acquires the identification code CX2, the identification code CX3, and the identification code CX4 from the database 41, and acquires the identification code CY1 from the database 42.

The information comparison processing unit 52 compares between the identification codes acquired by the identification code information acquisition unit 51. For example, at step S104 in FIG. 2, the information comparison processing unit 52 compares the identification code CX1 with the identification code CY1, to determine the consistency of the subjects. Moreover, at step S110 in FIG. 2, the information comparison processing unit 52 compares the identification code CX2, the identification code CX3, and the identification code CX4 with the identification code CY1, to determine the consistency of the subjects. The results of the comparison by the information comparison processing unit 52 are displayed on the display 60.

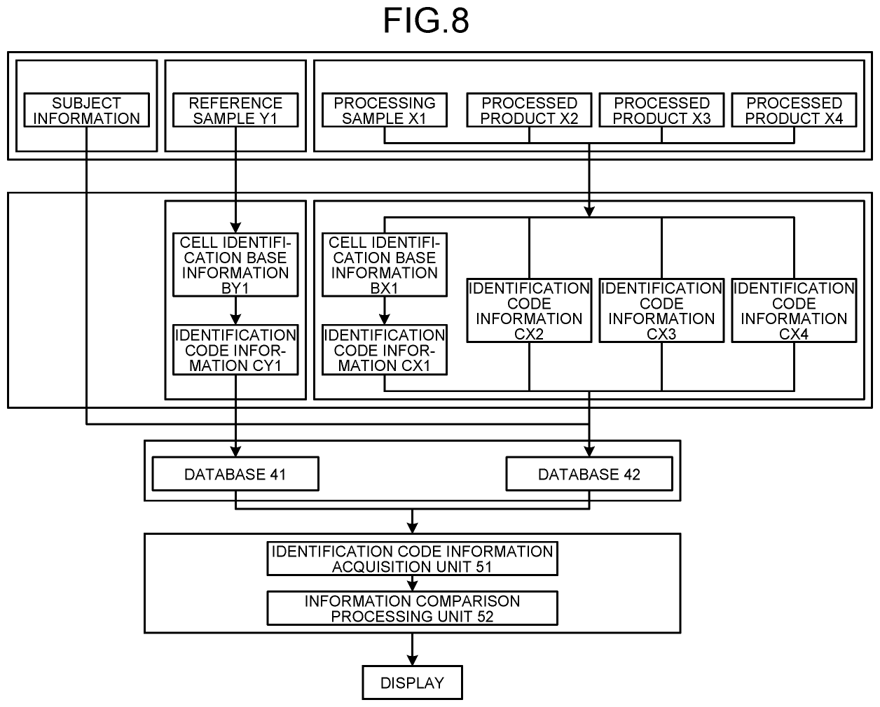

Next, the processing of the cell management system 1 will be described with reference to the flowchart in FIG. 8. First, various types of information such as the subject information, sample information, and processed product information are input via the input terminal 10 or the like, and are registered in the database 41 and the database 42. Moreover, the processing sample X1 and the reference sample Y1 are collected from the subject. Moreover, based on the processing sample X1, the processed product X2, the processed product X3, and the processed product X4 will be produced. Upon receiving the inputs of the subject information, sample information, processed product information, processing sample X1, reference sample Y1, processed product X2, processed product X3, and processed product X4, processing of the cell management system 1 will be performed. These inputs may be made on different days. For example, the processing sample X1 and the reference sample Y1 may be collected in separate tests.

The analysis apparatus 20 obtains the cell identification base information BX1 from the processing sample X1, and obtains the identification code information CX1. Moreover, the analysis apparatus 20 obtains the identification code information CX2 from the processed product X2. Furthermore, the analysis apparatus 20 obtains the identification code information CX3 from the processed product X3. Still furthermore, the analysis apparatus 20 obtains the identification code information CX4 from the processed product X4. The cell identification base information BX1, the identification code information CX1, the identification code information CX2, the identification code information CX3, and the identification code information CX4 are stored in the database 41. Moreover, the analysis apparatus 20 obtains the cell identification base information BY1 from the reference sample Y1, and obtains the identification code information CY1. The cell identification base information BY1 and the identification code information CY1 are stored in the database 42.

The identification code information acquisition unit 51 acquires the identification codes from the database 41 and the database 42 as appropriate, and the information comparison processing unit 52 compares between the identification codes acquired by the identification code information acquisition unit 51. The results of the comparison by the information comparison processing unit 52 are displayed on the display 60.

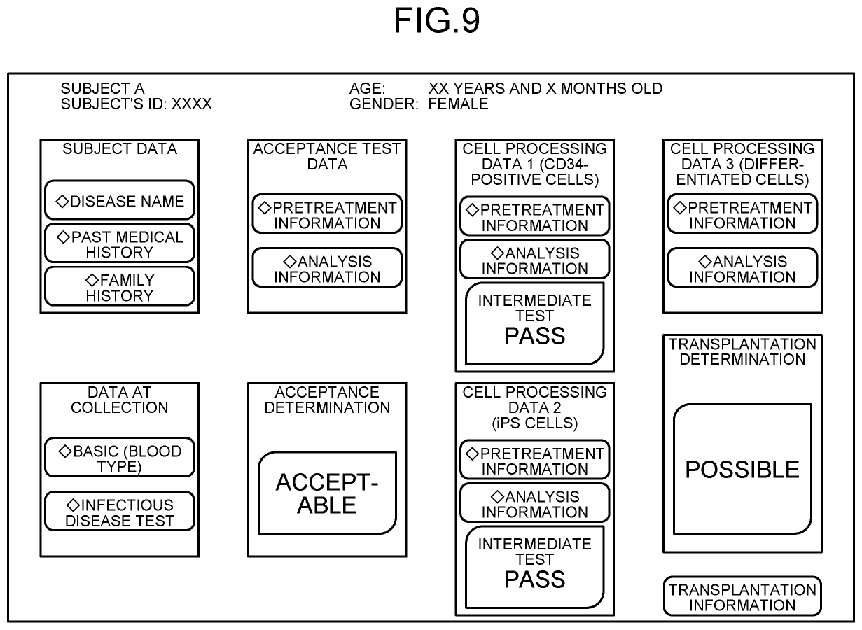

Next, a display example of the display 60 will be described with reference to FIG. 9. FIG. 9 is a display example corresponding to step S111 in FIG. 2, for example.

For example, the display 60 displays subject information such as the subject's name, subject's ID, age, and gender. Moreover, the display 60 displays more detailed subject information on the “Subject Data” tab. For example, if the user selects one of the icons of “Disease Name”, “Past Medical History”, “Family History”, and the like, the display 60 will display the detailed subject information contained in the selected icon.

Furthermore, the display 60 displays sample information on the “Data at Collection” and “Acceptance Test Data” tabs. For example, if the user selects one of the icons of “Basic (blood type)”, “Infectious Disease Test”, “Pretreatment Information”, “Analysis Information”, and the like, the display 60 will display the detailed sample information contained in the selected icon.

Still furthermore, the display 60 displays the determination results of the consistency of the subjects performed by the determination apparatus 50 based on the identification code CX1 and the identification code CY1, on the “Acceptance Determination” tab. In FIG. 9, the determination apparatus 50 determines the “same”, and the display 60 displays “Acceptable”.

Moreover, the display 60 displays processed product information on each processed product. For example, the display 60 displays the processed product information on the processed product X2, on the “Cell Processing Data 1 (CD34-positive cells)” tab. For example, if the user selects one of the icons of “Pretreatment Information”, “Analysis Information”, and the like, the display 60 displays the detailed processed product information contained in the selected icon. Furthermore, the display 60 displays the determination results of the consistency of the subjects performed by the determination apparatus 50 based on the identification code CX2 and the identification code CY1, on the “Intermediate Test” tab. In FIG. 9, the determination apparatus 50 determines the “same”, and the display 60 displays “Pass”.

Still furthermore, the display 60 displays the processed product information on the processed product X3, on the “Cell Processing Data 2 (iPS cells)” tab. For example, if the user selects one of the icons of “Pretreatment Information”, “Analysis Information”, and the like, the display 60 displays the detailed processed product information contained in the selected icon. Still furthermore, the display 60 displays the determination results of the consistency of the subjects performed by the determination apparatus 50 based on the identification code CX3 and the identification code CY1, in the “Intermediate Test” tab. In FIG. 9, the determination apparatus 50 determines the “same”, and the display 60 displays “Pass”.

Still furthermore, the display 60 displays the processed product information on the processed product X4, on the “Cell Processing Data 3 (differentiated cells)” tab. For example, if the user selects one of the icons of “Pretreatment Information”, “Analysis Information”, and the like, the display 60 displays the detailed processed product information contained in the selected icon.

Still furthermore, the display 60 displays the determination results of the consistency of the subjects performed by the determination apparatus 50 based on the identification code CX2, the identification code CX3, the identification code CX4, and the identification code CY1, on the “Transplantation Determination” tab. In FIG. 9, the determination apparatus 50 determines the “same”, and the display 60 displays that transplantation is “possible”.

The display in FIG. 9 can be sequentially updated in each of the steps illustrated in FIG. 2. For example, at the stage of obtaining the subject information at step S101, the display 60 only displays the subject information such as the subject's name, subject's ID, age, and gender, and the “Subject Data” tab. After the sample information is entered at step S102, the display 60 can display additional tabs of “Data at Collection” and “Acceptance Test Data”. After the acceptance determination is made at step S106, the display 60 can display an additional tab of “Acceptance Determination”. After the processed product information is entered at step S107, the display 60 can display additional tabs of “Cell Processing Data 1 (CD34-positive cells)”, “Cell Processing Data 2 (iPS cells)”, and “Cell Processing Data 3 (differentiated cells)”. After the transplantation is determined at step S112, the display 60 can display an additional tab of “Transplantation Determination”. Moreover, after the transplantation is carried out at step S113, the display 60 can display an additional tab of “Transplantation Information”. The tabs to be displayed may be changed as appropriate according to the input operations from the user.

Examples of producing the CD34-positive cells, iPS cells, and cardiomyocytes have been described. However, the embodiment is not limited thereto. For example, the embodiment can be similarly applied to the case such as the self-repair in spinal injury and cerebral infarction using autologous mesenchymal stem cells and muse cells, and the self-repair in neurodegeneration using specifically differentiated cells derived from autologous iPS cells.

Moreover, the embodiment is not limited to the case where the processed product is transplanted to the subject. For example, the embodiment can be similarly applied to the case where a prior drug response evaluation and a disease response evaluation are performed. In the prior drug response evaluation and the disease response evaluation, research has been developed on the prior drug evaluation and physical constitution evaluation using autologous iPS cells. Specifically, the research includes drug sensitivity evaluation using specifically differentiated cells (for example, cellular response during exposure to anticancer agents) and disease susceptibility studies using specifically differentiated cells (for example, QT prolongation during COVID-19 progression). The embodiments described above can be applied to any other example where the cell management is required.

For example, the term “processor” used in the above description refers to a circuit such as a CPU, a Graphics Processing Unit (GPU), an Application Specific Integrated Circuit (ASIC), a programmable logic device (for example, a Simple Programmable Logic Device (SPLD), a Complex Programmable Logic Device (CPLD), and a Field Programmable Gate Array (FPGA)). For example, if the processor is a CPU, the processor reads and executes a computer program stored in a storage circuit to implement the function. On the other hand, for example, if the processor is an ASIC, instead of storing a computer program in the storage circuit, the function is directly embedded in a circuit of the processor as a logic circuit. Each processor in the embodiment is not limited to being configured as a single circuit for each processor, but may also be configured as a single processor by combining a plurality of independent circuits to implement the functions. Furthermore, a plurality of the components in each drawing may be integrated into a single processor to implement the functions.

Moreover, the computer program to be executed by the processing circuit may be stored in a single memory, or a plurality of memories may be distributed and arranged, and a corresponding computer program may be read from individual memory. Still furthermore, instead of storing a computer program in the memory, the computer program can be directly incorporated into the circuit of the processor. In this case, the processor reads and executes the computer program embedded in the circuit to implement the function.

The components of each device according to the above embodiments are functionally conceptual, and need not necessarily be physically configured as illustrated. That is, the specific mode of dispersion and integration of each device is not limited to the ones illustrated in the drawings, and all or a part thereof can be functionally or physically dispersed or integrated in an optional unit, depending on various kinds of load and the status of use. Moreover, all or an optional part of each processing function carried out in each device can be realized by a CPU and a computer program analyzed and executed by the CPU, or may be realized as hardware by the wired logic.

Furthermore, the cell management method described in the above embodiments may be realized by executing a computer program prepared in advance on a computer such as a personal computer or workstation. This computer program can be distributed via a network such as the Internet. Moreover, this computer program can be recorded on a non-transitory computer-readable recording medium such as a hard disk, flexible disk (FD), CD-ROM, MO, and DVD, and executed by being read from the recording medium by a computer.

According to at least one embodiment described above, it is possible to implement simple and reliable cell management.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A cell management system, comprising:
an analysis apparatus configured to acquire a first identification code from a processing sample from which a cell used for treatment is produced, or from a processed product based on the processing sample, and to acquire a second identification code from a reference sample; and
a determination apparatus, based on the first identification code and the second identification code, configured to determine consistency of a subject from whom the processing sample is obtained and a subject from whom the reference sample is obtained.

2. The cell management system according to claim 1, wherein the analysis apparatus acquires at least one of a short tandem repeat sequence, a multi-locus minisatellite sequence, a single-locus minisatellite sequence, and a mitochondrial DNA sequence, as the first identification code and the second identification code.

3. The cell management system according to claim 1, wherein
the analysis apparatus acquires a plurality of the first identification codes from a plurality of the processed products, and
the determination apparatus determines the consistency, based on the first identification codes based on the processed products, and the second identification code.

4. The cell management system according to claim 1, wherein the processing sample and the reference sample are different types of samples obtained from a same subject.

5. The cell management system according to claim 1, wherein the processing sample and the reference sample are same type of samples obtained from a same subject on different tests.

6. A cell management method, comprising:
acquiring a processing sample from which a cell used for treatment is produced;
acquiring a reference sample;
acquiring a first identification code from the processing sample, or from a processed product based on the processing sample;
acquiring a second identification code from a reference sample; and
based on the first identification code and the second identification code, determining consistency of a subject from whom the processing sample is obtained and a subject from whom the reference sample is obtained.
